(19) United States
(12) Patent Application Publication
(10) Pub. No.: US 2006/0104921 A1
(43) Pub. Date: May 18, 2006

(54) Novel gum delivery systems and methods of making thereof

(76) Inventors: John A. Kostinko, Bel Air, MD (US); Andrew L. Oltmans, Alexandria, VA (US); Kathleen S. Pike, Bel Air, MD (US)

Correspondence Address: William S. Parks, 333 Thornall Street, Edison, NJ, 08837, US

(21) Appl. No.: 11/320,187

(22) Filed: Dec. 28, 2005

Related U.S. Application Data
(63) Continuation of application No. 10/428,957, filed on May 2, 2003.

(51) Int. Cl. A61K 8/73

(52) U.S. Cl. 424/49

(57) ABSTRACT

A substrate having about 0.5 wt % to about 25 wt % gum incorporated upon it is provided that provides the benefit of delayed viscosity when incorporated into a person care composition such as a toothpaste. Also disclosed is a method for forming gum incorporated onto or into a substrate comprising the steps of: providing a substrate; introducing a liquefied solution of gum to the substrate; and optionally drying the gum-treated substrate to form a dried gum-treated substrate. The invention further includes a dentifrice comprising a gum-treated substrate, and one or more ingredients selected from the group consisting of humectants, abrasives, thickening agents, binders, stabilizing agents, antibacterial agents, fluorides, sweeteners, and surfactants.

### CROSS-REFERENCE TO RELATED APPLICATIONS

This is a continuation of pending U.S. application Ser. No. 10/428,957, filed May 2, 2003, the content of which is incorporated herein by reference in its entirety for all purposes.

### BACKGROUND OF THE INVENTION

In the manufacturing of consumer products such as a toothpaste, rheology control agents such as gums are often used for several different reasons, such as to provide a gelatinous structure that stabilizes the toothpaste against phase separation, as well as to provide paste firmness and extrudability, and improved mouthfeel. However, although highly functional in a toothpaste formulation, the use of such gums creates numerous manufacturing difficulties. Notably, adding gum agents can cause non-uniform dispersion, bring about high dust levels and cause lumping. These problems can be addressed by sufficiently hydrating the gums, but such hydration can be a time-consuming process, and result in a significant reduction in toothpaste production rates.

Yet another difficulty is that the combination of gums with certain toothpaste ingredients such as humectants, abrasives, thickeners and results in a substantial degree of viscosity build as soon as these ingredients are mixed together. In fact, viscosity in the range of 200,000 cps is not uncommon, which can itself cause numerous problems for the throughput of the toothpaste plant. These problems include: (1) longer mixing times associated with the difficulty of blending the abrasive and/or thickener into the viscous toothpaste base; (2) the creation of lumps in the toothpaste as a result of high viscosity and inadequate shear; and (3) the difficulty of blending the dry powder formulation into a highly viscous toothpaste base.

Accordingly the manufacturing process itself must be adjusted to make accommodations for the aforementioned problems. For example toothpaste strainers (which remove lumps and entrained air) must be installed and operated under high pressures because of the toothpaste viscosity. Additionally, toothpaste tube filling time is slowed because of the high viscosity and significant pressure needed to force the viscous product into the tubes. Thus, the high initial toothpaste viscosity results in increased mixing time, increased time to strain the product, and an increased time to fill the tubes. These problems can be yet further exacerbated when the toothpaste cannot be added to tubes immediately and the toothpaste must sit in a holding vessel for an extended period of time, allowing the viscosity of the toothpaste to continue to increase, making eventual tubing of the product and cleaning of the holding vessel more difficult.

Given the foregoing, there is a need to develop a method for delivering gum into a consumer product formulation (e.g., in the form of a gum-treated substrate) that will address gum dispersion and hydration time, as well as the high viscosity issues of compounding toothpaste and thus, allow for improved throughput and increased production rates.

### BRIEF SUMMARY OF THE INVENTION

The invention includes a substrate having about 0.5 wt % to about 25 wt % gum incorporated thereon.

The invention also includes a method for forming gum incorporated onto or into a substrate comprising the steps of: providing a substrate; introducing a liquefied solution of gum to the substrate; and optionally drying the gum-treated substrate to form a dried gum-treated substrate.

The invention also includes a dentifrice comprising a gum-treated substrate, and one or more ingredients selected from the group consisting of humectants, abrasives, thickening agents, binders, stabilizing agents, antibacterial agents, fluorides, sweeteners, and surfactants.

### DETAILED DESCRIPTION OF THE INVENTION

All parts, percentages and ratios used herein are expressed by weight unless otherwise specified. All documents cited herein are incorporated by reference. The following describes preferred embodiments of the present invention, which provides a delayed viscosity build toothpaste using gum-treated substrates. While the optimal use for this gum-treated substrate is in dentifrices, it may also be used in a variety of other consumer products.

By “coated” it is meant that the specified coating ingredient covers at least a portion of the outer surface of a particle or substrate.

By “mixture” it is meant any combination of two or more substances, in the form of, for example without intending to be limiting, a heterogeneous mixture, a suspension, a solution, a sol, a gel, a dispersion, or an emulsion.

By “dentifrices” it is meant oral care products such as, without intending to be limiting, toothpastes, tooth powders, chewing gums and denture creams.

By “gum” it is meant any polysaccharide or derivative thereof that will hydrate in water to form viscous solutions, dispersions, or gels.

By “liquefied” it is meant to put in a liquid state by either melting or solubilizing in a solvent such as water.

By the “storage period” of a product it is meant the period of time between (a) the completion of manufacturing the product, and (b) the first use of the product by the consumer.

By “storing”, it is meant any manufacturing steps (including mere storage) during the period of time between (a) the completion of manufacturing the product, and (b) the first use of the product by the consumer.

The present invention relates to a gum-treated substrate for personal care products, that provides for the rapid dispersion of gum thickeners or gum binders into finished consumer products such as, without intending to be limiting, personal care products, food, pharmaceuticals, and cosmetics. The gum-treated substrate is particularly useful in dentifrices such as toothpastes.

For the embodiments of this gum-treated substrate in which gum is impregnated into a porous substrate (discussed in more detail below) offer the particular advantage of delaying viscosity build so that the personal care composition can be more easily processed. For example toothpastes containing gum-treated substrates, in which the gum is impregnated into the substrate, have a delayed viscosity build, of about 20,000 cps, approximately one-tenth of the viscosity value that would be expected. Because of this low initial viscosity, certain processing difficulties such as the long mixing times to incorporate dry powders, lump formation in the toothpaste due to the high viscosity, long or high-pressure strainer times, and slow tube filling operations are avoided. By avoiding these processing difficulties, production rates in existing dentifrice manufacturing facilities can be increased while investment costs for new facilities can be reduced because it is no longer necessary to install equipment to provide higher pressures in order to process high-viscosity toothpaste.

The gum-treated substrate itself is a mixture of at least a substrate and a gum thickener or gum binder. Preferably a liquefied (dissolved or melted) gum thickener or binder is either impregnated into a porous substrate or, alternately, coated onto a non-porous substrate. Any suitable porous or non-porous substrate can be used. By adding gum in the form of a gum-treated substrate, the gum rapidly and uniformly disperses in the product formulation. Delivery times are dramatically shortened, no lumping is experienced, mixing times are significantly reduced, time required to process the product through a strainer is greatly reduced, less product is lost to lumps collected on the strainer, and one experiences lower dust levels than when handling gums in their dry form. The use of a substrate to deliver the gums in a dry form also eliminates the time and expense associated with the use of a “make-down” and delivery system in which the gums are dissolved in water prior to use.

As discussed above, the additional benefit of delayed viscosity build can be realized when the gum is impregnated into the substrate. This delayed viscosity occurs because when the gum is impregnated into a porous substrate, the gum does not initially react with the other components of the consumer product (such as a toothpaste), and time is required for the gum to migrate out of the substrate pores and into to the toothpaste, so that the initial viscosity of the product remains relatively low, and thus, the product can be easily processed. This lower viscosity has a significant effect on every aspect of processing: mixing times are shorter, pumping rates can be increased, filtration/strainer rates are significantly increased, and packaging rates are dramatically increased. All of these improvements result to increase plant capacity. With time, the gum migrates from the substrate pores and into the product, so that the product gradually attains the desired level of viscosity in its final packaged form. Aging times can be regulated to produce the desired delayed viscosity build over a period of time by varying several different parameters, such as: the loading level of gum on the substrate, the amount of substrate incorporated in the formulation, and the gum type.

As mentioned above, the present invention relates to a gum-treated substrate which includes at least two components: a substrate and a gum. Substrates can be any finely divided, water-insoluble material preferably with an average particle size of 1 μm to about 850 μm (20 mesh), which is the about the smallest size visible to the naked eye, more preferably from about 5 μm to about 15 μm. Larger particle sizes can also be used if not objectionable to the formulation aesthetics, such as in facial scrub formulations where large, visible abrasives are preferred. The substrate used is limited only by its compatibility with the formulation into which it is delivered. For example, CMC could be delivered to a toothpaste batch by dicalcium phosphate dihydrate, TiO₂, alumina, sodium aluminosilicate, PCC, GCC, clay, or silica since all of these substrates are commonly found in toothpaste formulations. Likewise, CMC could be delivered on a substrate of apricot seed powder or walnut shell powder in facial scrub formulations and to food on a silica, spice or grain substrate.

The substrate may be either porous or non-porous. Preferable non-porous substrates include dicalcium phosphate dihydrate and calcium carbonate abrasives, titanium dioxide opacifier/colorant. As for the porous substrate, preferred materials include amorphous precipitated silica, silica gel or sodium aluminosilicate.

With regard specially to the aforementioned silicas, dental grade silica (amorphous precipitated silica, silica gel or silicon dioxide) and sodium aluminosilicate have a pore structure capable of absorbing significant amounts of liquids while still retaining their dry, free flowing character. These silicas or silicates may have relatively high absorption capacity (thickener grade silicas) or relatively low absorption capacity (abrasive grade silicas).

As mentioned before, gum thickening agents are useful in the dentifrice compositions of the present invention to provide a gelatinous structure that stabilizes the toothpaste against phase separation, provide paste firmness and extrudability, and improved mouthfeel. Commercially, they are usually referred to as thickeners, binders or stabilizers and are classified as natural or modified. Any gum suitable for use in toothpaste may be used as long as it can be either taken into solution or melted to allow for incorporation into the silica pores. Natural gums include carrageenan, gum tragacanth, gum karaya, gum arabic, gum ghatti, gum acacia, locust bean gum, sodium alginate, seaweed extracts, plant exudates, gums from seed or roots, and those obtained by microbial fermentation, such as Xanthan gum. Modified gums include cellulose, starch derivatives, and certain synthetic gums such as low methoxyl pectin, polyethylene glycol (PEG), propylene glycol, carboxymethyl, hydroxyethyl, hydroxypropyl, hydroxymethyl carboxyethyl, hydroxymethyl carboxypropyl, methyl, ethyl and sulfated cellulose; and guar gum. Lists are provided of gums used in cosmetic products in the International Cosmetics Ingredient Dictionary and Handbook, Seventh Edition, CTFA, Washington, D.C. 1997; in food and feed products in The Code of Federal Regulations, Title 21, (21 CFR), particularly sections 172, 184 and 582; and in pharmaceutical products in the United States Pharmacopeia and National Formulary (USP25/NF20), U.S. Pharmacopeial Convention, Inc., Rockville, Md., 2002, which are incorporated herein by reference.

Preferred gums are include Viscarin carrageenan-based products available from FMC Biopolymers, Rockland, Me., carboxymethyl cellulose-based products available from Hercules Corporation, Wilmington, Del., and Noviant, Inc, Nijmegen, the Netherlands and Xanthan-based products available from Jungbunzlauer, Basel, Switzerland and Rhodia Corporation, Cranbury, N.J.

As for the process of making the gum-treated substrate of the present invention, preferably the gum component is liquefied by dissolution in water or melting, and then absorbed into the substrate portion of the formulation. The substrate is then dried, if necessary to remove the introduced water, leaving behind a gum, which is now a solid and residing in the pores of the substrate. Specifically, in this process, a gum is liquefied by adding the gum to water at a concentration of about 0.1 wt % to about 20 wt %, preferably from about 0.5 wt % to about 10 wt %, and heating the mixture from about 20° C. to about 95° C., preferably from about 70° C. to about 90° C., until all the gum is solubilized. Alternately, the gum is liquefied by heating the gum to a temperature high enough to melt the gum, but not so high as to char the gum. The temperature used will depend on the melting point of the chosen gum. The liquefied gum is thereafter slowly added with agitation to a substrate over a period of about 5 to 10 minutes in a heated mixer in an amount to provide about 5 wt % to about 30 wt %, preferably about 20 wt % to about 25 wt %, gum on the substrate. The heated mixer is maintained at temperature sufficient to maintain the gum in a liquefied state, but not so high as to char the gum. The actual gum addition time is dependent on the size of the prepared batch.

Next, the gum-treated substrate is optionally dried to remove excess water at a temperature of generally not more than 105° C. Again, the drying temperature is dependent on the particular gum used. For example, xanthan and CMC gum-treated substrates can be dried at 105° C. without charring, while carrageenan gum-treated substrate is dried at about 75° C., since it chars at 105° C. Drying is not required when the gum has been melted or the water-gum solution is added at such a rate as to maintain the substrate in a dry and free-flowing state. The resulting product may be treated multiple times with the gum solution. The final product may be lightly milled and screened to remove lumps.

The gum-treated substrate may then be incorporated into a consumer product such as a dentifrice composition, e.g., a toothpaste. When used in a dentifrice or toothpaste, the gum-treated substrate serves as a thickening agent (which are also sometimes known as binders or stabilizing agents) or combination thickener/abrasive. Along with the additive the dentifrice or toothpaste may also contain several other ingredients such as abrasives, other thickeners, humectants, antibacterial agents, fluorides, flavors, sweeteners, and co-surfactants.

Abrasives impart improved cleaning and abrasive characteristics when included within a toothpaste or dentifrice. Abrasives clean teeth by removing debris and residual stains and function to polish tooth surfaces. Precipitated silicon dioxide, silica gel, precipitated calcium carbonate, ground calcium carbonate, chalk, sodium aluminosilicate and dibasic calcium phosphate dihydrate are examples of abrasives used in dentifrices. A sufficient amount of abrasive should be added to a toothpaste composition so that the radioactive dentin abrasion (RDA) value of the toothpaste is between about 50 and 200. Suitable abrasives can be generally added at a level of from about 5 wt. % to about 50 wt. %.

Optionally, additional thickeners (binders) may be incorporated into the dentifrice. This is particularly the case when the gum-treated substrate is not a thickener grade silica or silicate. These additional thickeners provide the dentifrice formulation with firmness, bulk, mouthfeel and thixotropy. The binders may be selected from inorganic thickeners such as precipitated silica, silica aerogel, pyrogenic silica, silicate clays and colloidal magnesium aluminum silicate and synthetic organic polymers such as polyacrylates and polyvinyl pyrrolidone, and mixtures thereof. These binders generally comprise about 5% to about 10% of the formulation, by weight.

Humectants serve to add body or “mouth texture” to a dentifrice as well as preventing the dentifrice from drying out. Suitable humectants include glycerin (glycerol), sorbitol, polyalkylene glycols such as polyethylene glycol and polypropylene glycol, hydrogenated starch hydrolyzates, xylitol, erythritol, mannitol, lactitol, hydrogenated corn syrup, and other edible polyhydric alcohols, used singly or as mixtures thereof. Suitable humectants can be added generally at a level of from about 15% to about 70%.

Suitable antimicrobial agents, (cationic, anionic and nonionic) are contemplated by the invention. Antimicrobial agents may be included to reduce the presence of microorganisms to below known harmful levels. Suitable antimicrobial agents include bisguanides, such as alexidine, chlorhexidine and chlorhexidine gluconate; quaternary ammonium compounds, such as benzalkonium chloride (BZK), benzethonium chloride (BZT), cetylpyridinium chloride (CPC), and Domiphen bromide; metal salts, such as zinc citrate zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination. If present, the level of antimicrobial agent is preferably from about 0.1 wt. % to about 5 wt. % of the toothpaste composition.

Flavoring agents optionally can be added to dentifrice compositions. Suitable flavoring agents include oil of Wintergreen, oil of peppermint, oil of spearmint, oil of sassafras, and oil of clove, cinnamon, anethole, menthol, and other such flavor compounds to add fruit notes, spice notes, etc. These flavoring agents consist chemically of mixtures of aldehydes, ketones, esters, phenols, acids, and aliphatic, aromatic and other alcohols.

Sweeteners may be added to the toothpaste composition to impart a pleasing taste to the product. Suitable sweeteners include saccharin (as sodium, potassium or calcium saccharin), cyclamate (as a sodium, potassium or calcium salt), acesulfane-K, thaumatin, neohisperidin dihydrochalcone, ammoniated glycyrrhizin, dextrose, levulose, sucrose, mannose, and glucose. Flavoring and sweetening agents are generally used in dentifrices at levels of from about 0.005% to about 2% by weight

The toothpaste will also preferably contain fluoride salts to prevent the development and progression of dental caries. Suitable fluoride salts include sodium fluoride, potassium fluoride, zinc fluoride, stannous fluoride, zinc ammonium fluoride, sodium monofluorophosphate, potassium monofluorophosphate, laurylamine hydrofluoride, diethylaminoethyloctoylamide hydrofluoride, didecyldimethylammonium fluoride, cetylpyridinium fluoride, dilaurylmorpholinium fluoride, sarcosine stannous fluoride, glycine potassium fluoride, glycine hydrofluoride, and sodium monofluorophosphate. Typical levels of fluoride salts are from about 0.1 wt. % to about 5 wt. %.

Surfactants may also be included as additional cleansing and foaming agents, and may be selected from anionic surfactants, zwitterionic surfactants, nonionic surfactants, amphoteric surfactants, and cationic surfactants. Anionic surfactants are preferred, such as metal sulfate salts, such as sodium lauryl sulfate.

The dentifrices disclosed herein may also a variety of additional ingredients such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, whitening agents and preservatives.

Finally, in the case of a dentifrice or toothpaste, water provides the balance of the composition in addition to the additives mentioned. The water is preferably deionized and free of impurities. The dentifrice will comprise generally from about 5% to about 60% of water, preferably from about 5% to 20%, by weight, of the toothpaste compositions.

The invention will now be described in more detail with respect to the following, specific, non-limiting examples.

### EXAMPLE 1

In Example 1, a gum-treated substrate suitable for use in dentifrices as well as other products for rapid gum dispersion and delayed viscosity build and containing carrageenan gum and a precipitated silica thickener were prepared according to the present invention.

Specifically, 10% Viscarin® carrageenan gum solution was prepared by adding 75 grams of Viscarin 389 gum available from FMC Biopolymers to 675 ml of water, mixing and heating the mixture to 88° C. (190° F.) until all of the gum had dissolved. In a Hobart mixer bowl heated to 85° C., 300 g of the Viscarin gum solution was added with mixing at a rate of 38 ml/min for 7.8 minutes to 200 g of Zeodent® 165 thickener grade precipitated silica available from J. M. Huber Corporation. The resulting product was dried at 85° C. in a Fisher 400 Series Isotemp Oven to remove excess water. The Zeodent 165 silica contained 13% by weight Viscarin gum on a dry basis within its pores.

212 g of this product was then added to a Hobart mixing bowl for a second treatment. 188 g of 10% Viscarin 389 solution preheated to 88° C. was added to the treated silica (Zeodent 165+13% Viscarin) in Hobart mixer bowl heated to 85° C. at a rate of 22 ml/min for 8.75 minutes. The resulting product was dried at 85° C. in a Fisher 400 Series Isotemp Oven to remove excess water. Example 1 gum treated silica was calculated to contain 20.1% by weight Viscarin gum on a dry basis within its pores.

### EXAMPLE 2

In Example 2, a 5% Viscarin 389 gum solution was prepared by adding 60 grams of Viscarin 389 gum to 1140 grams of water, mixing and heating the mixture to 49° C. (120° F.) until all of the gum had dissolved. The solution was heated to 85° C. and then 250 g were added to 200 g of Zeodent® 165 silica thickener with mixing in a Hobart mixer bowl heated to 85° C. over a period of 10 minutes. The silica was allowed to mix at 85° C. and additional solution was added very slowly over a period of 5 hours. The addition rate was adjusted to match the evaporation rate such that the powder always remained free flowing. Total gum solution addition to the 200 g of silica was 1200 g. The resultant Example 2 product was calculated to contain 22.2 wt % Viscarin 389 on a dry basis within its pores.

To demonstrate their efficacy in consumer products, the gum-treated silicas of Examples 1 and 2 were incorporated as powders into two different toothpaste compositions (numbers 1-2), which are set forth in Table I, below. The performance of these compositions was compared with the performance of the following control toothpaste compositions: Composition 3, which contains silica and Viscarin 389 gum added separately to the toothpaste composition; Composition 4, which contains no Viscarin gum and no silica; Composition 5 which contains Viscarin 389 gum, but no silica; and Composition 6 which contains silica but no Viscarin gum. The amounts of ingredients used in these formulations are given in Table I, below.

These toothpaste samples were prepared as follows. For Compositions 3 and 5, a first mixture was formed by combining glycerin and Viscarin 389 carrageenan gum and then stirring the first mixture until the components dissolved. (Note that this first mixture is not formed in the preparation of Compositions 1 and 2, because in those compositions the Viscarin gum is absorbed into the silica. Nor is it necessary for Compositions 4 and 6, because they contain no Viscarin gum.) A second mixture was then formed by combining the following ingredients in sequence: deionized water, tetrapotassium pyrophosphate, sodium saccharin, sodium monofluorophosphate, and then stirring until the components are dissolved. The first and second mixtures were then combined while stirring to form a “premix”. For compositions 1, 2, 4, and 6 only the second mixture applies and it serves as the premix.

The premix was placed into a Ross mixer (model 130LDM, Charles Ross & Co., Hauppauge, N.Y.). Dibasic calcium phosphate dihydrate abrasive, and Zeodent 165 silica thickener (compositions 3 and 6) or the gum-containing silicas prepared according to examples 1 and 2 (compositions 1 and 2) were added to the premix, and the mixture mixed without vacuum. Note that Compositions 4 and 5 contain no silica thickener so only dibasic calcium phosphate was added. Then 30 inches of vacuum was drawn and each toothpaste composition was mixed for 15 minutes, and then sodium lauryl sulfate and flavor was added. The resulting mixture was stirred for 5 minutes at a reduced mixing speed. The toothpaste composition prepared were sealed in plastic toothpaste tubes for storage and future testing. The six different toothpaste compositions contained the ingredients in the amounts given in grams in Table I, below.

TABLE I
Toothpaste Compositions

| | Toothpaste Composition Number | | | | | |
|---|---|---|---|---|---|---|
| Ingredients | 1 | 2 | 3 | 4 | 5 | 6 |
| Glycerin, 99.5% | 22.000 | 22.000 | 22.000 | 22.000 | 22.000 | 22.000 |
| Viscarin Gum | 0.000 | 0.000 | 1.500 | 0.000 | 1.500 | 0.000 |
| Deionized Water | 22.264 | 22.933 | 20.764 | 29.690 | 28.090 | 22.167 |
| Tetrapotassium | 0.500 | 0.500 | 0.500 | 0.500 | 0.500 | 0.500 |
| Pyrophosphate | | | | | | |
| Sodium Saccharin | 0.200 | 0.200 | 0.200 | 0.200 | 0.200 | 0.200 |
| Sodium | 0.760 | 0.760 | 0.760 | 0.760 | 0.760 | 0.760 |
| Monofluorophosphate | | | | | | |
| Dibasic Calcium Phosphate | 45.000 | 45.000 | 45.000 | 45.000 | 45.000 | 45.000 |
| dihydrate abrasive | | | | | | |
| Example 1 product | 7.426 | 0.000 | 0.000 | 0.000 | 0.000 | 0.000 |
| Example 2 product | 0.000 | 6.757 | 0.000 | 0.000 | 0.000 | 0.000 |
| Zeodent 165 silica thickener | 0.000 | 0.000 | 7.426 | 0.000 | 0.000 | 7.426 |
| Sodium Lauryl Sulfate | 1.200 | 1.200 | 1.200 | 1.200 | 1.300 | 1.300 |
| Flavor | 0.650 | 0.650 | 0.650 | 0.650 | 0.650 | 0.650 |

After toothpaste compositions 1-6 were prepared as above, viscosity measurements were made on each as follows.

The toothpaste viscosity was measured using a Synchro-Lectric Model RVT Brookfield viscometer with Helipath Stand Model D. All measurements were made with spindle TF at 5 rpm at a temperature of 25° C. The initial viscosity of each toothpaste composition was measured and thereafter viscosity measurements were made at the following times: 24 hours, 48 hours, 72 hours, 1 week, 2 weeks, and 3 weeks.

The results of the Brookfield viscosity measurements are set forth in Table II, below. The viscosity units are expressed in cps×10⁴.

TABLE II
Viscosity of Toothpaste compositions measured over three weeks

| Tooth- | | | | | | | |
|---|---|---|---|---|---|---|---|
| paste | Viscosity, cps × 10⁴ | | | | | | |
| Compo- | | 24 | 48 | 72 | 1 | 2 | 3 |
| sition | Initial | Hours | Hours | Hours | Week | Weeks | Weeks |
| 1 | 9.5 | 16.5 | 21.0 | 26.0 | 28.5 | 44.0 | 55.0 |
| 2 | 1.6 | 2.5 | 3.8 | 4.0 | 10.0 | 14.0 | 20.0 |
| 3 | >200 | >200 | >200 | >200 | >200 | >200 | >200 |
| 4 | <0.1 | <0.1 | <0.1 | <0.1 | <0.1 | <0.1 | <0.1 |
| 5 | 33 | 36.5 | 33 | 32.7 | 37.3 | 36.3 | 48.5 |
| 6 | 32 | 32 | 31.3 | 35.2 | 54.5 | 68 | 85 |

Toothpaste compositions 1 and 2 contained the gum-treated silicas prepared in Examples 1 and 2, which were prepared according to the present invention. As can be seen in Table II, both offered a delayed viscosity build in the toothpaste compositions. Compositions 1 and 2 had a lower initial viscosity allowing for easier compounding of the toothpaste compositions while providing a gradual or delayed increase in the viscosity of the final toothpaste over time to a viscosity seen in a toothpaste prepared in the conventional manner.

Toothpaste compositions 3-6 were control compositions representing the prior art. Toothpaste composition 3 contained Viscarin 389 carrageenan gum and Zeodent 165 precipitated silica added to the formulation separately. Unlike the gum treated silicas of the present invention used in compositions 1 and 2, toothpaste composition 3 had high viscosity values from the beginning, which resulted in compounding difficulty. Toothpaste composition 4 contained no Viscarin 389 gum and no Zeodent 165 precipitated silica, resulting in a toothpaste having viscosity values that were much too low for toothpaste compositions. Toothpaste composition 5 contained Viscarin 389 carrageenan gum but no Zeodent 165 precipitated silica. Toothpaste composition 6 contained Zeodent 165 precipitated silica but no Viscarin 389 carrageenan gum. Both toothpaste composition 5 and 6 had acceptable viscosity values; however, the initial viscosity of each composition was extremely high, which would present compounding challenges avoided by Toothpaste Compositions 1 and 2.

### EXAMPLES 3-10

In Examples 3-10, gum treated substrates suitable for use in dentifrices as well as other products for rapid gum dispersion, were prepared according to the present invention.

In these examples an aqueous solution of gum is prepared by adding the gum to water at 50° C. and mixing. The hot gum solution was slowly added to a specified carrier and mixed utilizing a Kitchen Aid ProLine Mixer (model KSM5). The resultant product was dried in a Fisher 400 Series Isotemp Oven set at 105° C. to a moisture content of less than 10%. The dried product was thereafter lightly milled utilizing a Bantam-Mikro Pulverizer Micro Sample Mill Type CF to eliminate small lumps and reduce the powder back to the original particle size of the substrate used in the test. The powder was then screened through a 20 mesh screen to a size of less than (850 μm) to insure that all lumps had been removed. The gums used were Xanthan gum FS available from Jungbunzlauer, Aqualon CMC 7MXF available from Hercules Corporation and Viscarin 389 Carrageenan gum available from FMC Biopolymers. The substrates used were Zeodent 113 amorphous precipitated silica abrasive and Zeodent 165 amorphous precipitated thickener silica and Zeodent 012 amorphous precipitated sodium aluminosilicate abrasive all of which are available from J.M. Huber Corporation; ground calcium carbonate (GCC) available from Omya Corporation, Superior, Ariz.; titanium dioxide grade S available from American International Chemical, Natick, Mass.; and dicalcium phosphate dihydrate (DCPD) available from Rhodia Corporation, Cranbury, N.J. The amounts and types of ingredients used to prepare Example 3-10 are given in Table III below.

TABLE III
Parameters for Examples 3-10

| | | Gum | Gum | | | % Gum |
|---|---|---|---|---|---|---|
| | | Solution | Soln. | | Substrate | on |
| Example | Gum Type | Conc., % | amount, g | Substrate | wt., g | Substrate |
| 3 | Xanthan | 0.5 | 300 | Zeodent 165 Silica | 200 | 0.744 |
| 4 | Carrageenan | 4.5 | 300 | Zeodent 165 Silica | 200 | 6.323 |
| 5 | CMC | 3.0 | 125 | DCPD | 500 | 0.744 |
| 6 | CMC | 3.0 | 125 | GCC | 500 | 0.744 |
| 7 | CMC | 3.0 | 125 | TiO2 | 500 | 0.754 |
| 8 | CMC | 3.0 | 327.3 | Zeodent 113 Silica | 400 | 2.396 |
| 9 | CMC | 3.0 | 300 | Zeodent 165 Silica | 200 | 4.306 |
| 10 | CMC | 3.0 | 327.3 | Zeodent 012 sodium | 400 | 2.391 |
| | | | | aluminosilicate | | |

After being prepared as set forth above, the gum treated substrates of Examples 3-10 were subjected to rapid dispersion testing. Three control powders were also included in the rapid dispersion testing. Control 1 was Aqualon 7MXF carboxymethyl cellulose (CMC) gum available from Hercules, Control 2 was Xanthan Gum FS available from Jungbunzlauer, and Control 3 was Viscarin 389 carrageenan gum available from FMC Biopolymers. Controls 1-3 were gums containing no substrate.

Rapid dispersion testing was accomplished by adding the test sample to a 70% sorbitol solution (Sorbo by Ruger Chemical). The test was run at room temperature. A Lightnin Model MS 3000 laboratory mixer operating at 433 RPM provided agitation. The entire test sample was added to the sorbitol solution over a delivery time of 60 to 90 seconds. A Syntron Magnetic Feeder model FTO controlled delivery time. The sample and sorbitol were mixed until no visual lumps appeared in the slurry. The slurry was then poured over a 20-mesh (850 μm) screen to check for lumps. After 5 minutes, any lumps retained on the 20-mesh screen were weighed.

The results of the rapid dispersion testing for Examples 3-10 and the three controls are contained in Table IV below.

TABLE IV
Samples for and Results of Rapid Dispersion Testing

| | | | | | | | Mix | | |
|---|---|---|---|---|---|---|---|---|---|
| | | | | | | | Time To | | |
| | | | | | | | No | Lump | |
| | | | | | | Delivery | Visual | Wt. On | |
| | | | | Sorbitol | Sample | Time, | Lumps, | 20 Mesh | % Gum |
| Example | Substrate | Gum Type | Gum % | Wt., g | Wt., g | Sec | Min. | Screen, g | Added |
| Control 1 | None | CMC | 100 | 580 | 1.5 | 60 | >60 | 3.6 | 0.259 |
| Control 2 | None | Xanthan | 100 | 580 | 0.75 | 60 | >60 | 10.8 | 0.129 |
| Control 3 | None | Carrageenan | 100 | 580 | 1.5 | 60 | >60 | 4.7 | 0.259 |
| 3 | Zeodent | Xanthan | 0.744 | 290 | 100.8 | 60 | 5 | 0 | 0.129 |
| | 165 | | | | | | | | |
| 4 | Zeodent | Carrageenan | 6.323 | 580 | 23.7 | 77 | 5 | 0 | 0.259 |
| | 165 | | | | | | | | |
| 5 | DCPD | CMC | 0.744 | 580 | 201.5 | 80 | 7 | 0 | 0.259 |
| 6 | GCC | CMC | 0.744 | 580 | 201.5 | 60 | 7 | 0 | 0.259 |
| 7 | TiO₂ | CMC | 0.754 | 580 | 198.9 | 90 | 10 | 0 | 0.259 |
| 8 | Zeodent | CMC | 2.396 | 580 | 62.6 | 67 | 2 | 0 | 0.259 |
| | 113 | | | | | | | | |
| 9 | Zeodent | CMC | 4.306 | 580 | 34.8 | 80 | 3 | 0 | 0.259 |
| | 165 | | | | | | | | |
| 10 | Zeodent | CMC | 2.391 | 580 | 62.7 | 70 | 5 | 0 | 0.259 |
| | 012 | | | | | | | | |

As can be seen in the table, examples 3-10, which were prepared according to the present invention and were gum-treated substrates, performed substantially better than the control samples 1-3, which included only gum. For all of the gum-treated substrates of examples 3-10, when the substrate was mixed with sorbitol, the initial lumps formed in the mixture quickly dissipate (taking no more than 10 minutes, by visual inspection). By contrast, for all of control samples 1-3, time to dissipation was much greater, at least sixty minutes or more. This indicates that the samples prepared according to the present invention all contribute to a significantly less viscous final product, than the samples prepared according to the prior art.

Similarly, after the sorbitol/substrate mixtures of examples 3-10 were formed and deposited on the 20 mesh screen, there was no lump residue left on the 20 mesh screen. By contrast, all of mixtures that contained control samples 1-3 had lump residue left on the 20 mesh screen after five minutes had elapsed. This again indicates that the samples prepared according to the present invention all contribute to a significantly less viscous final product, than the samples prepared according to the prior art.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of forming a gum-treated substrate comprising the steps of:
a) liquefying a gum; and
b) applying the gum to a substrate.

2. The method of claim 1, wherein in step a) the gum is liquefied by mixing the gum with water to form a first mixture.

3. The method of claim 2, wherein the first mixture has a gum concentration of about 0.1 wt % to about 20 wt %.

4. The method of claim 3, wherein the water has a temperature of about 20° C. to about 95° C.

5. A method of providing a dentifrice comprising the steps of:
a) forming a gum-treated substrate according to the method of claim 1;
b) mixing the gum-treated substrate with one or more ingredients selected from the group consisting of abrasives, other thickeners, humectants, antibacterial agents, fluorides, flavors, sweeteners, and surfactants, to form a dentifrice, wherein a first viscosity measurement of the dentifrice is less than about 1.6×10⁴ cps; and
c) storing the dentifrice for a period of time, so that a second viscosity measurement of the dentifrice after the period of time is greater than about 10×10⁴ cps.
